(19) United States
(12) Patent Application Publication
(10) Pub. No.: US 2002/0026043 A1
(43) Pub. Date: Feb. 28, 2002

(54) DESERT HEDGEHOG RELATED NUCLEIC ACIDS AND PROTEINS

(76) Inventor: BRIAN TAYLOR EDMONDS, CARMEL, IN (US)

Correspondence Address: ELI LILY AND COMPANY, LILLY CORPORATE CENTER, PATENT DIVSION DC: 1104, INDIANAPOLIS, IN, 46285

(21) Appl. No.: 09/244,466

(22) Filed: Feb. 4, 1999

Related U.S. Application Data
(60) Provisional application No. 60/073,878, filed on Feb. 6, 1998.
(60) Provisional application No. 60/091,843, filed on Jul. 6, 1998.
(60) Provisional application No. 60/076,553, filed on Mar. 2, 1998.

(51) Int. Cl. A01N 37/18; A61K 38/00; C07H 21/04; C12N 15/00; C12N 15/09; C12N 15/63; C12N 15/70; C12N 15/74; C07K 5/00; C07K 7/00; C07K 16/00; C07K 17/00; C12N 5/00; C12N 5/02

(52) U.S. Cl. 536/23.5; 435/320.1; 435/325; 514/2; 530/324

(57) ABSTRACT

The invention provides isolated nucleic acid, proteins and peptides, wherein said proteins and peptides are related to the Hedgehog family of proteins. Also provided are vectors and transformed host cells for expressing said proteins, and a method for identifying compounds that bind and/or modulate the activity of said proteins, and pharmaceutical compositions and methods for treating spinal cord injury, male infertility, and tumor growth.

### CROSS-REFERENCE

This application claims the benefit of U.S. Provisional Application No. 60/073,878, filed Feb. 6, 1998; No. 60/076,553, filed Mar. 2, 1998; and No. 60/091,843, filed Jul. 6, 1998.

### BACKGROUND OF THE INVENTION

This invention relates to recombinant DNA technology. In particular the invention pertains to a Hedgehog-related nucleic acids from a human source, as well as proteins and/or peptides encoded thereby, and related thereto. Also contemplated are methods for identifying compounds that bind said proteins, and methods for treating a variety of abnormal conditions in mammals including humans such as male infertility, tumor growth, and spinal regeneration.

The hedgehog genes are found in many vertebrate and invertebrate species (See generally, M. Hammerschmidt, A. Brook, and A. McMahon, “The world according to hedgehog” Trends in Genetics, 13, 14-21, 1997). Members of the hedgehog gene family comprise secreted signaling molecules that control a variety of developmental processes. In invertebrates, hedgehog proteins are involved in pattern segmentation; in vertebrates hedgehog related proteins are involved in left-right asymmetry, polarity (in the central nervous system, somites, and limbs), organogenesis, chondrogeneis, and spermatogenesis. The progenitor hedgehog sequence (designated hh), first identified in Drosophila melanogaster, affects embryonic and larval development. Later investigations have revealed homologs to the hh sequence in vertebrates, including mammals. One hh homolog, termed Sonic hedgehog (Shh), has been found in mouse, human, rat, Xenopus, chicken and Zebrafish (See e.g. Chang et.al. Development, 120, 3339-53, 1994; Ekker et.al. Development, 121, 23337-47, 1995; Krauss et.al. Cell, 75, 1431-44, 1993; Riddle et.al. Cell, 75, 1401-16, 1993); another hh homolog, termed Indian hedgehog (Ihh), has been found in mouse, human, and chicken; a third homolog, termed Desert hedgehog (Dhh), has been found thus far only in mouse; a fourth homolog, termed Banded hedgehog (X-bhh), has been found only in Xenopus; a fifth, termed Cephalic hedgehog (X-chh), has been found so far only in Xenopus.

One member of the hedgehog gene family, Desert hedgehog (Dhh), is essential to the development of testis in mouse, and in spermatogenesis (Echelard et.al. Cell, 75, 1417-30, 1993; Bitgood and McMahon, Dev. Biol. 172, 126-38, 1995; Bitgood et.al. Curr. Biol. 6, 298-04, 1996). Dhh may also play an important role in the development and maintenance of the peripheral nervous system, and in the development and maintenance of cardiac function. In this regard, the Dhh gene expresses both in Schwann cells and the developing atrioventricular valve of the heart (See e.g. Bitgood & McMahon, Develop. Biol. 172, 126-38 (1995).

Disclosed herein is a human homolog of Dhh (termed “Dhh-H”), that expresses in the dorsal root ganglia of adult spinal cord and in testis. The invention disclosed herein relates to the Dhh-H gene and protein, and to the use of Dhh-H protein and related molecules for regenerating spinal cord in a mammal in need thereof for treating or inhibiting tumor growth and in the treatment of male infertility.

### BRIEF SUMMARY OF THE INVENTION

The present invention provides isolated expressed sequence tags (ESTs) and nucleic acid molecules that encode a human desert hedgehog protein, termed herein “Dhh-H”. Having Dhh-H ESTs and the full length gene sequence enables the production of recombinant Dhh-H protein, the isolation of orthologous genes from other organisms, and/or paralogous genes from the same organism, chromosome mapping studies, and the implementation of large scale screens to identify compounds that bind said Dhh-H, or related molecule, as a means to identify potential pharmaceutical compounds. The proteins and peptides described herein are also useful, when administered in therapeutically-effective amounts, for stimulating spinal cord regeneration in a patient in need thereof, in treating male infertility, and in treating or inhibiting tumor growth.

In one embodiment the present invention relates to Dhh-H protein, and protein or peptide molecules that are structurally and/or functionally related thereto.

In another embodiment the present invention relates to an isolated nucleic acid molecule encoding Dhh-H protein, and related proteins described herein.

In another embodiment, the present invention relates to a nucleic acid that hybridizes to SEQ ID NO:3, SEQ ID NO:4 or SEQ ID NO:5 under high stringency conditions, and encodes a protein that is neurotrophic and/or capable of inducing spinal cord regeneration, and/or in treating male infertility and/or in inhibiting or treating tumor growth.

In another embodiment, the present invention relates to an isolated protein or peptide comprising SEQ ID NO:2 or fragment thereof.

In a still further embodiment, the present invention relates to a method for treating cancer, and/or in preventing or inhibiting tumor growth, and/or in causing shrinkage of a cancerous tumor, in vivo or in vitro, and or in promoting spinal cord regeneration.

In yet another embodiment the present invention relates to a method for treating male infertility comprising the step of administering a therapeutically effective amount of desert hedgehog protein or peptide.

### DETAILED DESCRIPTION OF THE INVENTION

### Definitions

The term “analog” or “functional analog” refers to a modified form of Dhh-H in which at least one amino acid substitution has been made such that said analog retains substantially the same biological activity as the unmodified Dhh-H in vivo and/or in vitro.

The terms “complementary” or “complementarity” as used herein refer to the capacity of purine and pyrimidine nucleotides to associate through hydrogen bonding to form double stranded nucleic acid molecules. The following base pairs are related by complementarity: guanine and cytosine; adenine and thymine; and adenine and uracil. As used herein, “complementary” means that the aforementioned relationship applies to substantially all base pairs comprising two single-stranded nucleic acid molecules over the entire length of said molecules. “Partially complementary” refers to the aforementioned relationship in which one of two single-stranded nucleic acid molecules is shorter in length than the other such that a portion of one of the molecules remains single-stranded.

“Expressed sequence tag” (viz. EST) refers to a fragment of a full-length cDNA sequence. EST fragments can encode active or inactive peptide fragments and are useful, among other things, as nucleic hybridization probes.

“Fragment thereof” refers to a fragment, piece, or sub-region of a nucleic acid or protein molecule whose sequence is disclosed herein, such that said fragment comprises 5 or more amino acids, or 10 or more nucleotides that are contiguous in the parent protein or nucleic acid molecule from which said fragment derives.

“Functional fragment” as used herein, refers to an isolated sub-region, or fragment of a protein or peptide comprising a functionally distinct region such as an active site of an enzyme, or a ligand binding site, or any other site that produces or is responsible for a biological effect in vivo or in vitro. Functional fragments may be produced by cloning technology, or as the natural products of alternative splicing mechanisms.

“Functionally related” as used herein is applied to proteins or peptides that are predicted to be functionally similar or identical to a progenitor molecule, for example, Dhh-H or fragment thereof. Functionally related species are identified based on chemical and physical similarities in amino acid composition and sequence.

“Host cell” refers to any eucaryotic or procaryotic cell that is suitable for propagating and/or expressing a cloned gene contained on a vector that is introduced into said host cell by, for example, transformation or transfection, or the like.

Dhh-H refers to a gene or RNA (e.g. SEQ ID NO:1 or residues 180 through 1367 of SEQ ID NO:1, or the RNA equivalent thereof; viz. “U” replaces “T”) and a protein (SEQ ID NO:2) or fragment thereof. Dhh-H is a member of the family of Hedgehog related molecules. This family of molecules is primarily responsible for development during embryogenesis and in tissue regeneration in adult tissues, including spinal cord. The Dhh-H protein comprises several functional domains. Residues 1 through 23 of SEQ ID NO:2 comprise a putative signal sequence; residues 24 through 198 comprise a cell-bound fragment produced upon autocatalysis; residues 199 through 396 comprise a soluble form of the protein that is released upon autocatalysis.

The term “homolog” or “homologous” describes the relationship between different nucleic acid molecules or amino acid sequences in which said sequences or molecules are related by partial identity or similarity at one or more blocks or regions within said molecules or sequences.

The term “hybridization” as used herein refers to a process in which a single-stranded nucleic acid molecule joins with a complementary strand through nucleotide base pairing. “Selective hybridization” refers to hybridization under conditions of high stringency. The degree of hybridization depends upon, for example, the degree of homology, the stringency of hybridization, and the length of hybridizing strands.

“Isolated nucleic acid compound” refers to any RNA or DNA sequence, however constructed or synthesized, which is locationally distinct from its natural location.

A “nucleic acid probe” or “probe” as used herein is a labeled nucleic acid compound which hybridizes with another nucleic acid compound. “Nucleic acid probe” means a single stranded nucleic acid sequence that will combine with a complementary or partially complementary single stranded target nucleic acid sequence to form a double-stranded molecule. A nucleic acid probe may be an oligonucleotide or a nucleotide polymer. A probe will usually contain a detectable moiety which may be attached to the end(s) of the probe or be internal to the sequence of the probe.

The term “orthologue” or “orthologous” refers to two or more genes or proteins from different organisms that exhibit sequence homology.

The term “paralogue” or “paralogous” refers to two or more genes or proteins within a single organism that exhibit sequence homology.

The term “plasmid” refers to an extrachromosomal genetic element. The plasmids disclosed herein are commercially available, publicly available on an unrestricted basis, or can be constructed from readily available plasmids in accordance with published procedures.

A “primer” is a nucleic acid fragment which functions as an initiating substrate for enzymatic or synthetic elongation of, for example, a nucleic acid molecule.

The term “promoter” refers to a nucleic acid sequence that directs transcription, for example, of DNA to RNA. An inducible promoter is one that is regulatable by environmental signals, such as carbon source, heat, or metal ions, for example. A constitutive promoter generally operates at a constant level and is not regulatable.

“Recombinant DNA cloning vector” as used herein refers to any autonomously replicating agent, including, but not limited to, plasmids and phages, comprising a DNA molecule to which one or more additional DNA segments can or have been incorporated.

The term “recombinant DNA expression vector” or “expression vector” as used herein refers to any recombinant DNA cloning vector, for example a plasmid or phage, in which a promoter and other regulatory elements are present thereby enabling transcription of an inserted DNA, which may encode a protein.

The term “stringency” refers to hybridization conditions. High stringency conditions disfavor non-homologous base pairing. Low stringency conditions have the opposite effect. Stringency may be altered, for example, by temperature and salt concentration.

“Low stringency” conditions comprise, for example, a temperature of about 37° C. or less, a formamide concentration of less than about 50%, and a moderate to low salt (SSC) concentration; or, alternatively, a temperature of about 50° C. or less, and a moderate to high salt (SSPE) concentration, for example 1M NaCl.

“High stringency” conditions comprise, for example, a temperature of about 42° C. or less, a formamide concentration of less than about 20%, and a low salt (SSC) concentration; or, alternatively, a temperature of about 65° C., or less, and a low salt (SSPE) concentration. For example, high stringency conditions comprise hybridization in 0.5 M NaHPO₄, 7% sodium dodecyl sulfate (SDS), 1 mM EDTA at 65° C. (Ausubel, F. M. et al. Current Protocols in Molecular Biology, Vol. I, 1989; Green Inc. New York, at 2.10.3).

“SSC” comprises a hybridization and wash solution. A stock 20X SSC solution contains 3M sodium chloride, 0.3M sodium citrate, pH 7.0.

“SSPE” comprises a hybridization and wash solution. A 1× SSPE solution contains 180 mM NaCl, 9 mM Na₂HPO₄, 0.9 mM NaH₂PO₄ and 1 mM EDTA, pH 7.4.

“Substantially pure,” used in reference to a peptide or protein, means separation from other cellular and non-cellular molecules, including other protein molecules. A substantially pure preparation would be about at least 85% pure; preferably about at least 95% pure. A “substantially pure” protein can be prepared by a variety of techniques, well known to the skilled artisan, including, for example, the IMAC protein purification method.

“Treating” as used herein describes the management and care of a patient for the purpose of combating the disease, condition, or disorder and includes the administration of a protein of the present invention to prevent the onset of the symptoms or complications, alleviating the symptoms or complications, or eliminating the disease, condition, or disorder. Treating as used herein includes the administration of the protein for cosmetic purposes. A cosmetic purpose seeks to control, for example, the weight of a mammal to improve bodily appearance.

The term “vector” as used herein refers to a nucleic acid compound used for introducing exogenous or endogenous DNA into host cells. A vector comprises a nucleotide sequence which may encode one or more protein molecules. Plasmids, cosmids, viruses, and bacteriophages, in the natural state or which have undergone recombinant engineering, are examples of commonly used vectors.

The various restriction enzymes disclosed and described herein are commercially available and the manner of use of said enzymes including reaction conditions, cofactors, and other requirements for activity are well known to one of ordinary skill in the art. Reaction conditions for particular enzymes were carried out according to the manufacturer's recommendation.

The Dhh-H gene encodes a novel protein that is related to the Hedgehog family of proteins. An exemplary Dhh-H gene is disclosed herein as SEQ ID NO:1, the coding region comprising nucleotide residues 180 through 1367 of SEQ ID NO:1. Those skilled in the art will recognize that owing to the degeneracy of the genetic code, numerous “silent” substitutions of nucleotide base pairs could be introduced into the sequence identified as SEQ ID NO:1 without altering the identity of the encoded amino acid(s) or protein product. All such substitutions are intended to be within the scope of the invention.

Also contemplated by the present invention are Dhh-H proteins, for example SEQ ID NO:2, functional fragments thereof, and functionally related molecules. Hedgehog precursor proteins undergo an internal autoproteolytic cleavage at a conserved sequence comprising Gly Cys Phe such that an approximately 19 kDa amino terminal and 26 kDa carboxy terminal fragment are produced. The amino terminal fragment remains bound to the cell surface while the carboxy terminal fragment is diffusible. The amino terminal fragment is responsible for hedgehog signaling activities in Drosophila and vertebrates, whereas the carboxyl fragment appears to carry the autocatalytic protease activity (Porter et.al., Nature, 374, 363-66, 1995). Functional fragments comprise sub-regions of Dhh-H that retain biological activity in vivo or in vitro, for example, the capacity to induce spinal cord regeneration or to inhibit tumor cell growth, or to treat male menopause. Exemplary functional fragments of Dhh-H comprise residues 1 through 198, 24 through 198, and 199 through 396 of SEQ ID NO:2.

Amino acid substitution modifications can be made in accordance with the following Table 1.

TABLE 1
Amino Acid Substitutibility

| ORIGINAL RESIDUE | EXEMPLARY SUBSTITUTIONS |
|---|---|
| ALA | SER |
| ARG | LYS |
| ASN | GLN; HIS |
| ASP | GLU |
| CYS | SER |
| GLN | ASN |
| GLU | ASP |
| GLY | PRO |
| HIS | ASN, GLN |
| ILE | LEU, VAL |
| LEU | ILE, VAL |
| LYS | ARG, GLN, GLU |
| MET | LEU, ILE |
| PHE | MET, LEU, TYR |
| SER | THR |
| THR | SER |
| TRP | TYR |
| TYR | TRP, PHE |
| VAL | ILE, LEU |

Functionally-related Analogs

Proteins and peptides having biological activities that are similar or identical to Dhh-H, in vivo or in vitro, are also contemplated by the present invention. Said proteins, while being functionally related, comprise amino acid sequences that differ from SEQ ID NO:2. The amino acid sequences of analogs of Dhh-H can be generated by deletion, insertion, inversion, and/or substitution of one or more amino acid residues in said Dhh-H, or any one of the peptides disclosed herein. Substitution analogs can generally be made by solid phase or recombinant techniques in which single or multiple conservative amino acid substitutions are made, for example, according to Table 1. Generally, in the case of multiple substitutions, it is prefered that less than thirty residues be changed in any given molecule, most preferably between one to ten residues are changed in any given molecule such that about between 90% to 99% of residues are identical with the sequence of SEQ ID NO:2 or appropriate functional sub-region therein; alternatively, such that about between 95% to 99% of residues are identical with SEQ ID NO:2 or subregion therein.

Fragments of Proteins

One embodiment of the instant invention provides fragments of the proteins disclosed that may or may not be biologically active. Such fragments are useful, for example, as an antigen for producing an antibody to said proteins.

Fragments of the proteins disclosed herein may be generated by any number of suitable techniques, including chemical synthesis of any portion of SEQ ID NO:2, proteolytic digestion of said proteins, or most preferably, by recombinant DNA mutagenesis techniques, well known to the skilled artisan. See. e.g. K. Struhl, “Reverse biochemistry: Methods and applications for synthesizing yeast proteins in vitro,” Meth. Enzymol. 194, 520-535. For example, in a preferred method, a nested set of deletion mutations are introduced into a nucleic acid (e.g. residues 180 through 1367 of SEQ ID NO:1) encoding Dhh-H such that varying amounts of the protein coding region are deleted, either from the amino terminal end, or from the carboxyl end of the protein molecule. This method can also be used to create internal fragments of the intact protein in which both the carboxyl and amino terminal ends are removed. Several appropriate nucleases can be used to create such deletions, for example Bal31, or in the case of a single stranded nucleic acid molecule, mung bean nuclease. For simplicity, it is preferred that the Dhh-H gene be cloned into a single-stranded cloning vector, such as bacteriophage M13, or equivalent. If desired, the resulting gene deletion fragments can be subcloned into any suitable vector for propagation and expression of said fragments in any suitable host cell.

Functional fragments of the proteins disclosed herein may be produced as described above, preferably using cloning techniques to engineer smaller versions of the intact gene, lacking sequence from the 5′ end, the 3′ end, from both ends, or from an internal site. Fragments may be tested for biological activity using any suitable assay, for example, the ability of a protein fragment to induce tissue differentiation in vivo or in vitro.

Gene Isolation Procedures

Those skilled in the art will recognize that the Dhh-H gene could be obtained by a plurality of recombinant DNA techniques including, for example, hybridization, polymerase chain reaction (PCR) amplification, or de novo DNA synthesis. (See e.g., T. Maniatis et al. Molecular Cloning: A Laboratory Manual, 2d Ed. Chap. 14 (1989)).

Methods for constructing cDNA libraries in a suitable vector such as a plasmid or phage for propagation in procaryotic or eucaryotic cells are well known to those skilled in the art. [See e.g. Maniatis et al. Supra]. Suitable cloning vectors are well known and are widely available.

The Dhh-H gene (e.g. residues 180 through 1367 of SEQ ID NO:1), or fragment thereof, can be isolated from a tissue in which said gene is expressed, for example, testis or spinal cord. In one method, mRNA is isolated, and first strand cDNA synthesis is carried out. A second round of DNA synthesis can be carried out for the production of the second strand. If desired, the double-stranded cDNA can be cloned into any suitable vector, for example, a plasmid, thereby forming a cDNA library. Oligonucleotide primers targeted to any suitable region of SEQ ID NO:1 can be used for PCR amplification of Dhh-H. See e.g. PCR Protocols: A Guide to Method and Application, Ed. M. Innis et al., Academic Press (1990). The PCR amplification comprises template DNA, suitable enzymes, primers, and buffers, and is conveniently carried out in a DNA Thermal Cycler (Perkin Elmer Cetus, Norwalk, Conn.). A positive result is determined by detecting an appropriately-sized DNA fragment following agarose gel electrophoresis.

Protein Production Methods

One embodiment of the present invention relates to the substantially purified protein encoded by the Dhh-H gene.

Skilled artisans will recognize that the proteins of the present invention can be synthesized by a number of different methods, such as chemical methods well known in the art, including solid phase peptide synthesis or recombinant methods. Both methods are described in U.S. Pat. No. 4,617,149, incorporated herein by reference.

The principles of solid phase chemical synthesis of polypeptides are well known in the art and may be found in general texts in the area. See, e.g., H. Dugas and C. Penney, Bioorganic Chemistry (1981) Springer-verlag, New York, 54-92. For example, peptides may be synthesized by solid-phase methodology utilizing an Applied Biosystems 430A peptide synthesizer (Applied Biosystems, Foster City, Calif.) and synthesis cycles supplied by Applied Biosystems.

The proteins of the present invention can also be produced by recombinant DNA methods using the cloned Dhh-H gene. Recombinant methods are preferred if a high yield is desired. Expression of the cloned gene can be carried out in a variety of suitable host cells, well known to those skilled in the art. For this purpose, the Dhh-H gene is introduced into a host cell by any suitable means, well known to those skilled in the art. While chromosomal integration of the cloned gene is within the scope of the present invention, it is preferred that the gene be cloned into a suitable extra-chromosomally maintained expression vector so that the coding region of the Dhh-H gene is operably-linked to a constitutive or inducible promoter.

The basic steps in the recombinant production of the Dhh-H protein are:

a) constructing a natural, synthetic or semi-synthetic DNA encoding Dhh-H protein;

b) integrating said DNA into an expression vector in a manner suitable for expressing the Dhh-H protein, either alone or as a fusion protein;

c) transforming or otherwise introducing said vector into an appropriate eucaryotic or prokaryotic host cell forming a recombinant host cell,

d) culturing said recombinant host cell in a manner to express the Dhh-H protein; and

e) recovering and substantially purifying the Dhh-H protein by any suitable means, well known to those skilled in the art.

Expressing Recombinant Dhh-H Protein in Procaryotic and Eucaryotic Host Cells

Procaryotes may be employed in the production of recombinant Dhh-H protein. For example, the Escherichia coli K12 strain 294 (ATCC No. 31446) is particularly useful for the prokaryotic expression of foreign proteins. Other strains of E. coli, bacilli such as Bacillus subtilis, enterobacteriaceae such as Salmonella typhimurium or Serratia marcescans, various Pseudomonas species and other bacteria, such as Streptomyces, may also be employed as host cells in the cloning and expression of the recombinant proteins of this invention.

Promoter sequences suitable for driving the expression of genes in procaryotes include b-lactamase [e.g. vector pGX2907, ATCC 39344, contains a replicon and b lactamase gene], lactose systems [Chang et al., Nature (London), 275:615 (1978); Goeddel et al., Nature (London), 281:544 (1979)], alkaline phosphatase, and the tryptophan (trp) promoter system [vector pATH1 (ATCC 37695)], which is designed to facilitate expression of an open reading frame as a trpE fusion protein under the control of the trp promoter. Hybrid promoters such as the tac promoter (isolatable from plasmid pDR540, ATCC-37282) are also suitable. Still other bacterial promoters, whose nucleotide sequences are generally known, may be ligated to DNA encoding the protein of the instant invention, using linkers or adapters to supply any required restriction sites. Promoters for use in bacterial systems also will contain a Shine-Dalgarno sequence operably-linked to the DNA encoding the desired polypeptides. These examples are illustrative rather than limiting.

The proteins of this invention may be synthesized either by direct expression or as a fusion protein comprising the protein of interest as a translational fusion with another protein or peptide which may be removable by enzymatic or chemical cleavage. It is often observed in the production of certain peptides in recombinant systems that expression as a fusion protein prolongs the life span, increases the yield of the desired peptide, or provides a convenient means of purifying the protein. This is particularly relevant when expressing mammalian proteins in procaryotic hosts. A variety of peptidases (e.g. enterokinase and thrombin) which cleave a polypeptide at specific sites or digest the peptides from the amino or carboxy termini (e.g. diaminopeptidase) of the peptide chain are known. Furthermore, particular chemicals (e.g. cyanogen bromide) will cleave a polypeptide chain at specific sites. The skilled artisan will appreciate the modifications necessary to the amino acid sequence (and synthetic or semi-synthetic coding sequence if recombinant means are employed) to incorporate site-specific internal cleavage sites. See e.g., P. Carter, “Site Specific Proteolysis of Fusion Proteins”, Chapter 13, in Protein Purification: From Molecular Mechanisms to Large Scale Processes, American Chemical Society, Washington, D.C. (1990).

In addition to procaryotes, a variety of amphibian expression systems such as frog oocytes, and mammalian cell systems can be used. The choice of a particular host cell depends to some extent on the particular expression vector used. Exemplary mammalian host cells suitable for use in the present invention include HepG-2 (ATCC HB 8065), CV-1 (ATCC CCL 70), LC-MK₂ (ATCC CCL 7.1), 3T3 (ATCC CCL 92), CHO-K1 (ATCC CCL 61), HeLa (ATCC CCL 2), RPMI8226 (ATCC CCL 155), H4IIEC3 (ATCC CCL 1600), C127I (ATCC CCL 1616), HS-Sultan (ATCC CCL 1484), and BHK-21 (ATCC CCL 10), for example.

A wide variety of vectors are suitable for transforming mammalian host cells. For example, the pSV2-type vectors comprise segments of the simian virus 40 (SV40) genome required for transcription and polyadenylation. A large number of plasmid pSV2-type vectors have been constructed, such as pSV2-gpt, pSV2-neo, pSV2-dhfr, pSV2-hyg, and pSV2-b-globin, in which the SV40 promoter drives transcription of an inserted gene. These vectors are widely available from sources such as the American Type Culture Collection (ATCC), 12301 Parklawn Drive, Rockville, Md., 20852, or the Northern Regional Research Laboratory (NRRL), 1815 N. University Street, Peoria, Ill., 61604.

Promoters suitable for expression in mammalian cells include the SV40 late promoter, promoters from eukaryotic genes, such as, for example, the estrogen-inducible chicken ovalbumin gene, the interferon genes, the glucocorticoid-inducible tyrosine aminotransferase gene, the thymidine kinase gene promoter, and the promoters of the major early and late adenovirus genes and the cytomegalovirus promoter.

Plasmid pRSVcat (ATCC 37152) comprises portions of a long terminal repeat of the Rous Sarcoma virus, a virus known to infect chickens and other host cells. This long terminal repeat contains a promoter which is suitable for use in the vectors of this invention. H. Gorman et al., Proc. Nat. Acad. Sci. (USA), 79, 6777 (1982). The plasmid pMSVi (NRRL B-15929) comprises the long terminal repeats of the Murine Sarcoma virus, a virus known to infect mouse and other host cells. The mouse metallothionein promoter has also been well characterized for use in eukaryotic host cells and is suitable for use in the present invention.

This promoter is present in the plasmid pdBPV-MMTneo (ATCC 37224) which can serve as the starting material for the construction of other plasmids of the present invention.

Transfection of mammalian cells with vectors can be performed by a plurality of well known processes including, but not limited to, protoplast fusion, calcium phosphate co-precipitation, electroporation and the like. See, e.g., Maniatis et al., supra.

Some viruses also make appropriate vectors. Examples include the adenoviruses, the adeno-associated viruses, the vaccinia virus, the herpes viruses, the baculoviruses, and the rous sarcoma virus, as described in U.S. Pat. No. 4,775,624, incorporated herein by reference.

Eucaryotic microorganisms such as yeast and other fungi are also suitable host cells. The yeast Saccharomyces cerevisiae is the preferred eucaryotic microorganism. Other yeasts such as Kluyveromyces lactis and Pichia pastoris are also suitable. For expression in Saccharomyces, the plasmid YRp7 (ATCC-40053), for example, may be used. See, e.g., L. Stinchcomb et al., Nature, 282, 39 (1979); J. Kingsman et al., Gene, 7, 141 (1979); S. Tschemper et al., Gene, 10, 157 (1980). Plasmid YRp7 contains the TRP1 gene which provides a selectable marker for use in a trp1 auxotrophic mutant.

Purification of Recombinantly-produced Dhh-H Protein

An expression vector carrying a cloned Dhh-H gene, or EST thereof, is transformed or transfected into a suitable host cell using standard methods. Cells that contain the vector are propagated under conditions suitable for expression of the recombinant Dhh-H protein. For Example, if the recombinant gene has been placed under the control of an inducible promoter, suitable growth conditions would incorporate the appropriate inducer. The recombinantly-produced protein may be purified from cellular extracts of transformed cells by any suitable means.

In a preferred process for protein purification, the Dhh-H gene is modified at the 5′ end to incorporate several histidine residues at the amino terminus of the Dhh-H protein. This “histidine tag” enables a single-step protein purification method referred to as “immobilized metal ion affinity chromatography” (IMAC), essentially as described in U.S. Pat. No. 4,569,794, which hereby is incorporated by reference. The IMAC method enables rapid isolation of substantially pure recombinant Dhh-H protein starting from a crude extract of cells that express a modified recombinant protein, as described above.

Production of Antibodies

The proteins of this invention and fragments thereof may be used in the production of antibodies. The term “antibody” as used herein describes antibodies, fragments of antibodies (such as, but not limited, to Fab, Fab′, Fab2′, and Fv fragments), and chimeric, humanized, veneered, resurfaced, or CDR-grafted antibodies capable of binding antigens of a similar nature as the parent antibody molecule from which they are derived. The instant invention also encompasses single chain polypeptide binding molecules.

The production of antibodies, both monoclonal and polyclonal, in animals, especially mice, is well known in the art. See, e.g., C. Milstein, Handbook of Experimental Immunology, (Blackwell Scientific Pub., 1986); J. Goding, Monoclonal Antibodies: Principles and Practice, (Academic Press, 1983). For the production of monoclonal antibodies the basic process begins with injecting a mouse, or other suitable animal, with an immunogen. The mouse is subsequently sacrificed and cells taken from its spleen are fused with myeloma cells, resulting in a hybridoma that reproduces in vitro. The population of hybridomas is screened to isolate individual clones, each of which secretes a single antibody species, specific for the immunogen. Each antibody obtained in this way is the clonal product of a single B cell.

Chimeric antibodies are described in U.S. Pat. No. 4,816,567, the entire contents of which is herein incorporated by reference. This reference discloses methods and vectors for the preparation of chimeric antibodies. An alternative approach is provided in U.S. Pat. No. 4,816,397, the entire contents of which is herein incorporated by reference. This patent teaches co-expression of the heavy and light chains of an antibody in the same host cell.

The approach of U.S. Pat. No. 4,816,397 has been further refined in European Patent Publication No. 0 239 400. The teachings of this European patent publication are a preferred format for genetic engineering of monoclonal antibodies. In this technology the complementarity determining regions (CDRs) of a human antibody are replaced with the CDRs of a murine monoclonal antibody, thereby converting the specificity of the human antibody to the specificity of a murine antibody.

Single chain antibodies and libraries thereof are yet another variety of genetically engineered antibody technology that is well known in the art. (See, e.g. R. E. Bird, et al., Science 242:423-426 (1988); PCT Publication Nos. WO 88/01649, WO 90/14430, and WO 91/10737. Single chain antibody technology involves covalently joining the binding regions of heavy and light chains to generate a single polypeptide chain. The binding specificity of the intact antibody molecule is thereby reproduced on a single polypeptide chain.

The antibodies contemplated by this invention are useful in diagnostics, therapeutics or in diagnostic/therapeutic combinations.

The proteins of this invention or suitable fragments thereof can be used to generate polyclonal or monoclonal antibodies, and various inter-species hybrids, or humanized antibodies, or antibody fragments, or single-chain antibodies. The techniques for producing antibodies are well known to skilled artisans. (See e.g. A. M. Campbell, Monoclonal Antibody Technology: Laboratory Techniques in Biochemsitry and Molecular Biology, Elsevier Science Publishers, Amsterdam (1984); Kohler and Milstein, Nature 256, 495-497 (1975); Monoclonal Antibodies: Principles & Applications Ed. J. R. Birch & E. S. Lennox, Wiley-Liss, 1995.

A protein used as an immunogen may be modified or administered in an adjuvant, by subcutaneous or intraperitoneal injection into, for example, a mouse or a rabbit. For the production of monoclonal antibodies, spleen cells from immunized animals are removed, fused with myeloma cells, such as SP2/0-Ag14 cells, and allowed to become monoclonal antibody producing hybridoma cells in the manner known to the skilled artisan. Hybridomas that secrete a desired antibody molecule can be screened by a variety of well known methods, for example ELISA assay, western blot analysis, or radioimmunoassay (Lutz et al. Exp. Cell Res. 175, 109-124 (1988); Monoclonal Antibodies: Principles & Applications Ed. J. R. Birch & E. S. Lennox, Wiley-Liss, 1995).

For some applications labeled antibodies are desirable. Procedures for labeling antibody molecules are widely known, including for example, the use of radioisotopes, affinity labels, such as biotin or avidin, enzymatic labels, for example horseradish peroxidase, and fluorescent labels, such as FITC or rhodamine (See e.g. Enzyme-Mediated Immunoassay, Ed. T. Ngo, H. Lenhoff, Plenum Press 1985; Principles of Immunology and Immunodiagnostics, R. M. Aloisi, Lea & Febiger, 1988).

Labeled antibodies are useful for a variety of diagnostic applications. In one embodiment the present invention relates to the use of labeled antibodies to detect the presence of Dhh-H. Alternatively, the antibodies could be used in a screen to identify potential modulators of Dhh-H. For example, in a competitive displacement assay, the antibody or compound to be tested is labeled by any suitable method. Competitive displacement of an antibody from an antibody-antigen complex by a test compound such that a test compound-antigen complex is formed provides a method for identifying compounds that bind Dhh-H.

The Dhh-H related nucleic acid molecules of the invention may be produced by chemical synthetic methods. The synthesis of nucleic acids is well known in the art. See, e.g., E. L. Brown, R. Belagaje, M. J. Ryan, and H. G. Khorana, Methods in Enzymology, 68:109-151 (1979). Fragments of the DNA sequence corresponding to the Dhh-H gene could be generated using a conventional DNA synthesizing apparatus, such as the Applied Biosystems Model 380A or 380B DNA synthesizers (Applied Biosystems, Inc., 850 Lincoln Center Drive, Foster City, Calif. 94404) using phosphoramidite chemistry, thereafter ligating the fragments so as to reconstitute the entire gene. Alternatively, phosphotriester chemistry may be employed to synthesize the nucleic acids of this invention. (See, e.g., M. J. Gait, ed., Oligonucleotide Synthesis, A Practical Approach, (1984)).

In an alternative methodology, namely PCR, the DNA sequences disclosed and described herein, comprising, for example, a portion or all of SEQ ID NO:1 can be produced from a plurality of starting materials. For example, starting with a cDNA preparation (e.g. cDNA library) derived from a tissue that expresses the Dhh-H gene, suitable oligonucleotide primers complementary to the coding region of SEQ ID NO:1, or to any sub-region therein, are prepared as described in U.S. Pat. No. 4,889,818, hereby incorporated by reference. Other suitable protocols for the PCR are disclosed in PCR Protocols: A Guide to Method and Applications, Ed. Michael A. Innis et al., Academic Press, Inc. (1990). Using PCR, any region of the Dhh-H gene can be targeted for amplification such that full or partial length gene sequences may be produced.

The ribonucleic acids of the present invention may be prepared using polynucleotide synthetic methods discussed supra, or they may be prepared enzymatically, for example, using RNA polymerase to transcribe a Dhh-H DNA template.

The most preferred systems for preparing the ribonucleic acids of the present invention employ the RNA polymerase from the bacteriophage T7 or the bacteriophage SP6. These RNA polymerases are highly specific, requiring the insertion of bacteriophage-specific sequences at the 5′ end of the template to be transcribed. See, Maniatis et al., supra.

This invention also provides nucleic acids, RNA or DNA, that are complementary to SEQ ID NO:1, or fragment thereof.

Nucleic Acid Probes

The present invention also provides probes and primers useful for a variety of molecular biology techniques including, for example, hybridization screens of genomic, subgenomic, or cDNA libraries, as well as hybridization against nucleic acids derived from cell lines or tissues that originate from drug-resistant tumors. Such hybridization screens are useful as methods to identify homologous and/or functionally related sequences from the same or other organisms. A nucleic acid compound comprising SEQ ID NO:1, SEQ ID NO:3, SEQ ID NO:4, or SEQ ID NO:5, or a complementary sequence thereof, or a fragment thereof, or an RNA counterpart thereof, which is at least 14 base pairs in length, and which will selectively hybridize to human DNA or mRNA encoding Dhh-H protein or fragment thereof, or a functionally related protein, is provided. Preferably, the 14 or more base pair compound is DNA. See e.g. B. Wallace and G. Miyada, “Oligonucleotide Probes for the Screening of Recombinant DNA Libraries,” In Meth. Enzym., 152, 432-442, Academic Press (1987).

Probes and primers can be prepared by enzymatic or recombinant methods, well known to those skilled in the art (See e.g. Sambrook et al. supra). A probe may be a single stranded nucleic acid sequence which is complementary in some particular degree to a nucleic acid sequence sought to be detected (“target sequence”). A probe may be labeled with a detectable moiety such as a radio-isotope, antigen, or chemiluminescent moiety. A description of the use of nucleic acid hybridization as a procedure for the detection of particular nucleic acid sequences is described in U.S. Pat. No. 4,851,330 to Kohne, entitled “Method for Detection, Identification and Quantitation of Non-Viral Organisms.”

Having the DNA sequence of the present invention allows preparation of relatively short DNA (or RNA) sequences having the ability to specifically hybridize to gene sequences disclosed herein. In these aspects, nucleic acid probes of an appropriate length are prepared. The ability of such nucleic acid probes to specifically hybridize to a polynucleotide encoding a Dhh-H gene or related sequence lends particular utility in a variety of embodiments. Most importantly, the probes may be used in a variety of assays for detecting the presence of complementary sequences in a given sample.

In certain embodiments, it is advantageous to use oligonucleotide primers. The sequence of such primers is designed using a polynucleotide of the present invention for use in detecting, amplifying or mutating a defined segment of a gene or polynucleotide that encodes a Dhh-H polypeptide using PCR technology.

Preferred nucleic acid sequences employed for hybridization studies, or assays, include probe molecules that are complementary to at least an about 14 to an about 70-nucleotide long stretch of a polynucleotide that encodes a Dhh-H polypeptide, such as the nucleotide base sequences designated as SEQ ID NO:1, SEQ ID NO:3, SEQ ID NO:4, or SEQ ID NO:5. A length of at least 14 nucleotides helps to ensure that the fragment is of sufficient length to form a duplex molecule that is both stable and selective. Molecules having complementary sequences over stretches greater than 14 bases in length are generally preferred, though in order to increase stability and selectivity of the hybrid. One will generally prefer to design nucleic acid molecules having gene-complementary stretches of 25 to 40 nucleotides, 55 to 70 nucleotides, or even longer where desired. Such fragments may be readily prepared by, for example, directly synthesizing the fragment by chemical means, by application of nucleic acid reproduction technology, such as the PCR TM technology of U.S. Pat. No. 4,603,102, herein incorporated by reference, or by excising selected DNA fragments from recombinant plasmids containing appropriate inserts and suitable restriction enzyme sites.

The following guidelines are useful for designing probes with desirable characteristics. The extent and specificity of hybridization reactions are affected by a number of factors that determine the sensitivity and specificity of a particular probe, whether perfectly complementary to its target or not. The affect of various experimental parameters and conditions are well known to those skilled in the art.

First, the stability of the probe:target nucleic acid hybrid should be chosen to be compatible with the assay conditions. This may be accomplished by avoiding long A and T rich sequences, by terminating the hybrids with G:C base pairs and by designing a probe with an appropriate Tm (i.e. melting temperature). The melting profile, including the Tm of a hybrid comprising an oligonucleotide and target sequence, may be determined using a Hybridization Protection Assay. The probe should be chosen so that the length and % GC content result in a Tm about 2°-10° C. higher than the temperature at which the final assay will be performed. The gobase composition of the probe is also a significant factor because G-C base pairs exhibit greater thermal stability as compared to A-T base pairs. Thus, hybridization involving complementary nucleic acids of higher G-C content will be more stable at higher temperatures.

The ionic strength and incubation temperature under which a probe will be used should also be taken into account. It is known that hybridization will increase as the ionic strength of the reaction mixture increases, and that the thermal stability of molecular hybrids will increase with increasing ionic strength. On the other hand, chemical reagents such as formamide, urea, DMSO and alcohols, which disrupt hydrogen bonds, increase the stringency of hybridization. Destabilization of hydrogen bonds by such reagents can greatly reduce the Tm. In general, optimal hybridization for synthetic oligonucleotide probes of about 10-50 bases in length occurs approximately 5° C. below the melting temperature for a given duplex. Incubation at temperatures below the optimum may allow mismatched base sequences to hybridize and can therefore result in reduced specificity.

The length of the target nucleic acid sequence and, accordingly, the length of the probe sequence can also be important. In some cases, there may be several sequences from a particular region, varying in location and length, which will yield probes with the desired hybridization characteristics. In other cases, one sequence may be significantly better than another even though the one sequence differs merely by a single base. Finally, there can be intramolecular and intermolecular hybrids formed within a probe if there is sufficient self-complementarity. Such structures can be avoided through careful probe design. Computer programs are available to search for this type of interaction.

A probe molecule may be used for hybridizing to a sample suspected of possessing a Dhh-H or Dhh-H-related nucleotide sequence. The hybridization reaction is carried out under suitable conditions of stringency.

Alternatively, such DNA molecules may be used in a number of techniques including their use as: (1) diagnostic tools to detect polymorphisms in DNA samples from a human or other mammal; (2) means for detecting and isolating homologs of Dhh-H and related polypeptides from a DNA library potentially containing such sequences; (3) primers for hybridizing to related sequences for the purpose of amplifying those sequences; and (4) primers for altering the native Dhh-H DNA sequences; as well as other techniques which rely on the similarity of the DNA sequences to those of the Dhh-H DNA segments herein disclosed.

Once synthesized, oligonucleotide probes may be labeled by any of several well known methods. See e.g. Maniatis et.al., Molecular Cloning (2d ed. 1989). Useful labels include radioisotopes, as well as non-radioactive reporting groups. Isotopic labels include H³, S³⁵, P³², I¹²⁵, Cobalt, and C¹⁴. Most methods of isotopic labeling involve the use of enzymes and include methods such as nick-translation, end-labeling, second strand synthesis, and reverse transcription. When using radio-labeled probes, hybridization can be detected by autoradiography, scintillation counting, or gamma counting. The detection method selected will depend upon the hybridization conditions and the particular radio isotope used for labeling.

Non-isotopic materials can also be used for labeling, and may be introduced internally into the sequence or at the end of the sequence. Modified nucleotides may be incorporated enzymatically or chemically, and chemical modifications of the probe may be performed during or after synthesis of the probe, for example, by the use of non-nucleotide linker groups. Non-isotopic labels include fluorescent molecules, chemiluminescent molecules, enzymes, cofactors, enzyme substrates, haptens or other ligands. In a preferred embodiment of the invention, the length of an oligonucleotide probe is greater than or equal to about 18 nucleotides and less than or equal to about 50 nucleotides. Labeling of an oligonucleotide of the present invention may be performed enzymatically using [³²P]-labeled ATP and the enzyme T4 polynucleotide kinase.

Vectors

Another aspect of the present invention relates to recombinant DNA cloning vectors and expression vectors comprising the nucleic acids of the present invention. The preferred nucleic acid vectors are those which comprise DNA. The most preferred recombinant DNA vectors comprise residues 180 through 1367 of SEQ ID NO:1.

The skilled artisan understands that choosing the most appropriate cloning vector or expression vector depends upon a number of factors including the availability of restriction enzyme sites, the type of host cell into which the vector is to be transfected or transformed, the purpose of the transfection or transformation (e.g., stable transformation as an extrachromosomal element, or integration into the host chromosome), the presence or absence of readily assayable or selectable markers (e.g., antibiotic resistance and metabolic markers of one type and another), and the number of copies of the gene desired in the host cell.

Vectors suitable to carry the nucleic acids of the present invention comprise RNA viruses, DNA viruses, lytic bacteriophages, lysogenic bacteriophages, stable bacteriophages, plasmids, viroids, and the like. The most preferred vectors are plasmids.

When preparing an expression vector the skilled artisan understands that there are many variables to be considered, for example, whether to use a constitutive or inducible promoter. The practitioner also understands that the amount of nucleic acid or protein to be produced dictates, in part, the selection of the expression system. Regarding promoter sequences, inducible promoters are preferred because they enable high level, regulatable expression of an operably-linked gene. The skilled artisan will recognize a number of suitable promoters that respond to a variety of inducers, for example, carbon source, metal ions, and heat. Other relevant considerations regarding an expression vector include whether to include sequences for directing the localization of a recombinant protein. For example, a sequence encoding a signal peptide preceding the coding region of a gene is useful for directing the extracellular export of a resulting polypeptide.

The present invention also provides a method for constructing a recombinant host cell capable of expressing peptides and/or proteins comprising Dhh-H, said method comprising transforming or otherwise introducing into a host cell a recombinant DNA vector that comprises an isolated DNA sequence that encodes Dhh-H. A suitable host cell is any eucaryotic cell that can accomodate high level expression of an exogenously introduced gene or protein, and that will incorporate said protein into its membrane structure. Vectors for expression are those which comprise SEQ ID NO:1, or fragment thereof. Transformed host cells may be cultured under conditions well known to skilled artisans such that Dhh-H or fragment thereof is expressed, thereby producing a recombinant Dhh-H protein in the recombinant host cell.

For the purpose of identifying compounds to treat male infertility, it would be desirable to identify compounds that bind the Dhh-H protein and/or modify its activity. A method for determining agents that bind the Dhh-H protein comprises contacting the Dhh-H protein with a test compound and monitoring binding by any suitable means.

The instant invention provides a screening system for discovering compounds that bind the Dhh-H protein, said screening system comprising the steps of:

a) preparing or purifying Dhh-H protein by any suitable means;

b) exposing said Dhh-H protein to a test compound;

c) quantifying the binding of said compound to Dhh-H protein by any suitable means.

Utilization of the screening system described above provides a means to determine compounds that may alter the biological function of Dhh-H. This screening method may be adapted to large-scale, automated procedures such as a PANDEX® (Baxter-Dade Diagnostics) system, allowing for efficient high-volume screening of potential therapeutic agents.

In such a screening protocol Dhh-H is prepared as described herein, preferably using recombinant DNA technology. A test compound is introduced into a reaction vessel containing the Dhh-H protein or fragment thereof. Binding of Dhh-H by a test compound is determined by any suitable means. For example, in one method radioactively-labeled or chemically-labeled test compound may be used. Binding of the protein by the compound is assessed, for example, by quantifying bound label versus unbound label using any suitable method. Binding of a test compound may also be carried out by a method disclosed in U.S. Pat. No. 5,585,277, which hereby is incorporated by reference. In this method, binding of a test compound to a protein is assessed by monitoring the ratio of folded protein to unfolded protein, for example by monitoring sensitivity of said protein to a protease, or amenability to binding of said protein by a specific antibody against the folded state of the protein.

The foregoing screening methods are useful for identifying a ligand of a Dhh-H protein, perhaps as a lead to a pharmaceutical compound for the treatment of cancer or for inhibiting tumor growth, or for treating male infertility. A ligand that binds Dhh-H, or related fragment thereof, or receptor thereof, is identified, for example, by combining a test ligand with Dhh-H protein under conditions that cause the protein to exist in both folded and unfolded states. If the test ligand binds the folded state of the protein, the relative amount of folded protein will be higher than in the case of a test ligand that does not bind the protein. The ratio of protein in the folded versus unfolded state is easily determinable by, for example, susceptibility to digestion by a protease, or binding to a specific antibody, or binding to chaperonin protein, or binding to any suitable surface.

Also contemplated are methods for expressing and identifying the activity of recombinant Dhh-H proteins in oocytes and embryos, said methods being generally known in the art. For example, expression of the recombinant protein(s) to be tested in the subject assay can be carried out by microinjection of cRNA encoding the Dhh-H protein, or fragment thereof, or by microinjection (or by other form of transfection) of an expression vector encoding the protein of interest. Either method can be carried out by employing the basics of expression cloning strategies known in the art. In one embodiment, cDNA libraries are cloned into vectors that can be used for in vitro RNA synthesis. For instance, the pCS2 +/− vector, which contains SP6, T7 and T3 promoters, may be introduced upstream and downstream of the cloning site in order to permit in vitro RNA synthesis upon linearization of the plasmid. In an illustrative embodiment, a plasmid containing the cDNA to be tested can be linearized by cutting downstream from the cDNA insert with a restriction enzyme. The post-restriction digest is digested with Proteinase K and then extracted with two phenol: chloroform (1:1) extractions. The resulting DNA fragments are then ethanol precipitated. The precipitated fragments are mixed with either T3 RNA polymerase (to make sense strand), or T7 RNA polymerase (to make anti-sense strand), plus rATP, rCTP, rGTP, rUTP, and RNase inhibitor. Simultaneously, capped RNA can be produced in vitro (Krieg and Melton, Meth Enzymol 155, 397-415, 1987; and Richardson et al. (1988) Bio/Technology 6:565-570). Other exemplary vectors useful in the subject assay include: the pSP64T vector (Kreig et al. (1984) Nuc Acid Res 12:7057-7071) which contains the SP6 promoter and the 5′ and 3′ untranslated flanking regions of Xenopus beta-globin cDNA to provide more stable RNA for translation in injectecd oocytes; the pOEV expression vector (Pfaff et al. (1990) Anal Biochem 188:192-199) which permits cloned DNA to be transcribed and translated directly in oocytes; and the pMT2 expression vector (Swick et al. (1992) PNAS 89:1812-1816).

In vitro effects of Dhh-H on primary cultures of Leydig cells from human or rodent testes or Leydig-derived cell lines can also be determined. Sertoli cells of the germinal epithelium in mouse testes express high levels of Dhh mRNA. See e.g. Bitgood and McMahon, Dev. Biol. 172, 126-138 (1995). Human testes also expresses high levels of Dhh-H in Sertoli cells. Leydig cells located in the neighboring interstitium appear to be targets for Dhh secretion from the Sertoli cells. The response of the Leydig cells is to increase the expression of specific genes that have been linked to exposure to Hh in other model systems. Assessment of the expression levels of these genes in Leydig cells before and after exposure to Dhh-H is contemplated. In addition, Leydig cells are the primary source of testosterone in the testes and treatment with Dhh is exprected to increase the production/secretion of testosterone.

The effects of reduced Dhh-H expression on Sertoli cell physiology is also of interest. The testes of male homozygous Dhh-H knockout mice are much reduced in size compared to heterozygous litter mates. While much of this can be attributed to reduced numbers of germ cells (spermatogonia), the loss of Dhh-H may also affect the growth and proliferation of Sertoli cells. The effects of Dhh on Sertoli cell growth is assessed directly in vivo or in vitro e.g. in either primary rat or mouse testes culture, or Sertoli-derived cell lines using antisense oligonucleotides directed against Dhh. Changes in Dhh mRNA expression correlate with changes in various physiological parameters; e.g., growth rate, secretory capacity and cell morphology.

In vitro effects of recombinant Dhh-H on neuronal survival and/or differentiation is also contemplated by the invention. Hh genes, in general, have been found to affect the differentiation of various neuronal populations. As Dhh has a very distinct neuronal expression pattern in mouse (Schwann cells enveloping peripheral and cranial nerves), it is of interest to assess the role of Dhh in the physiology of peripheral nerves. Two embodiments are contemplated. First, cultured explants from rat/mouse dorsal root ganglia (DRG) are grown in defined media with and without recombinant Dhh. Effects on neuronal growth rate, survival and morphology are observed. Second, co-cultures of mouse Schwann cells and DRG explants are implemented to study directly the effect of Dhh secreted by the Schwann cells. Antisense oligonucleotides directed against Dhh are used to modulate Dhh expression in the Schwann cells and the effects on DRG neuronal morphology/physiology can be studied.

Effects of recombinant Dhh on vascular endothelium proliferation and/or differentiation can also be studied. Dhh expression is observed in the endocardium of the heart and the endothelium of blood vessels. The effect of Dhh on the differentiation of the endothelium can be assessed using endothelial cell primary culture (obtained from placenta) or endothelial derived cell lines. The antisense strategy can also be used here.

The present invention also provides methods for treating humans and other mammals afflicted with diseases and conditions such as male infertility, tumor growth, and spinal cord injury, comprising administration of an effective amount of Dhh-H protein or functional fragment thereof, or analog thereof, in a dose of between 1 and 1000 ug/Kg bodyweight. For example, in practicing this method Dhh-h or related peptide or analog thereof (e.g. SEQ ID NO:2, or residues 24 through 396 of SEQ ID NO:2, or residues 199 through 396 of SEQ ID NO:2, a functional fragment(s) therof) can be administered in a single daily dose, in multiple doses per day, or by continuous or discontinuous administration via a mechanical pump device that is implanted in the body or otherwise attached thereto. The amount of Dhh-H or related protein or peptide to be administered will be determined by the physician, and depend on such factors as the nature and severity of the condition, syndrome, or disease being treated, and the age and general health of the patient.

The present invention also provides a pharmaceutical composition comprising as the active agent a Dhh-H polypeptide or fragment, or a pharmaceutically acceptable non-toxic salt thereof, or analog thereof, in admixture with a pharmaceutically acceptable solid or liquid carrier. The protein, preferably in the form of a pharmaceutically acceptable salt, can be formulated for parenteral administration for the therapeutic or prophylactic treatment of male infertility, male menopause, tumor growth, or spinal regeneration. For example, compounds of SEQ ID NO:2, or fragment thereof, can be admixed with conventional pharmaceutical carriers and excipients. The compositions comprising Dhh-H protein contain from about 0.1 to 90% by weight of the protein, preferably in a soluble form, and more generally from about 10 to 30%. Furthermore, the present proteins may be administered alone or in combination with other agents useful in treating such conditions.

For intravenous (IV) use, the Dhh-H protein, fragment, or analog is administered in commonly used intravenous fluid(s) and administered by infusion. Such fluids, for example, physiological saline, Ringer's solution or 5% dextrose solution can be used.

For intramuscular preparations, a sterile formulation, preferably a suitable soluble salt form of the Dhh-H protein, for example SEQ ID NO:2, (or residues 1 through 198, 24 through 198, and/or 199 through 396 of SEQ ID NO:2), such as the hydrochloride salt, can be dissolved and administered in a pharmaceutical diluent such as pyrogen-free water (distilled), physiological saline or 5% glucose solution. A suitable insoluble form of the compound may be prepared and administered as a suspension in an aqueous base or a pharmaceutically acceptable oil base, e.g. an ester of a long chain fatty acid such as ethyl oleate.

Skilled artisans will recognize that IC₅₀ values are dependent on the selectivity of the compound tested. For example, a compound with an IC₅₀ which is less than 10 nM is generally considered an excellent candidate for drug therapy. However, a compound which has a lower affinity, but is selective for a particular target, may be an even better candidate. The skilled artisan will recognize that any information regarding the binding potential, inhibitory activity, or selectivity of a particular compound is useful toward the development of pharmaceutical products.

The following examples more fully describe the present invention. Those skilled in the art will recognize that the particular reagents, equipment, and procedures described are merely illustrative and are not intended to limit the present invention in any manner.

### EXAMPLE 1

### RT-PCR Amplification of Dhh-H Gene from mRNA

A Dhh-H gene is isolated by reverse transcriptase PCR (RT-PCR) using conventional methods. Total RNA is prepared from a tissue that expresses the Dhh-H gene, for example testis, using standard methods. First strand cDNA synthesis is carried out using a commercially available kit (SuperScript™ System; Life Technologies) and primers directed at any suitable region of SEQ ID NO:1, for example, residues at or near 180 through residues at or near position 1367 of SEQ ID NO:1.

Amplification is carried out by adding to the first strand cDNA (dried under vacuum): 8 μl of 10× synthesis buffer (200 mM Tris-HCl, pH 8.4; 500 mM KCl, 25 mM MgCl₂, 1 ug/ul BSA); 68 μl distilled water; 1 μl each of a 10 uM solution of each primer; and 1 μl Taq DNA polymerase (2 to 5 U/μl). The reaction is heated at 94° C. for 5 min. to denature the RNA/cDNA hybrid. Then, 15 to 30 cycles of PCR amplification are performed using any suitable thermal cycle apparatus. The amplified sample may be analyzed by agarose gel electrophoresis to check for an appropriately-sized fragment.

### EXAMPLE 2

### Production of a Vector for Expressing Dhh-H in a Host Cell

An expression vector suitable for expressing Dhh-H or fragment thereof in a variety of procaryotic host cells, such as E. coli is easily made. The vector contains an origin of replication (Ori), an ampicillin resistance gene (Amp) useful for selecting cells which have incorporated the vector following a tranformation procedure, and further comprises the T7 promoter and T7 terminator sequences in operable linkage to a Dhh-H coding region. Plasmid pET11A (obtained from Novogen, Madison Wis.) is a suitable parent plasmid. PET11A is linearized by restriction with endonucleases NdeI and BamHI. Linearized pET11A is ligated to a DNA fragment bearing NdeI and BamHI sticky ends and comprising the coding region of the Dhh-H gene, as disclosed by Example 1, or fragment thereof.

The Dhh-H nucleic acid used in this construction may be slightly modified at the 5′ end (amino terminus of encoded protein) in order to simplify purification of the encoded protein product. For this purpose, an oligonucleotide encoding 8 histidine residues is inserted after the ATG start codon. Placement of the histidine residues at the amino terminus of the encoded protein serves to enable the IMAC one-step protein purification procedure.

### EXAMPLE 3

### Recombinant Expression and Purification of Dhh-H Protein

An expression vector that carries an ORF encoding Dhh-H or fragment thereof and which ORF is operably-linked to an expression promoter is transformed into E. coli BL21 (DE3) (hsdS gal 1cIts857 ind1Sam7nin5lacUV5-T7gene 1) using standard methods. Transformants, selected for resistance to ampicillin, are chosen at random and tested for the presence of the vector by agarose gel electrophoresis using quick plasmid preparations. Colonies which contain the vector are grown in L broth and the protein product encoded by the vector-borne ORF is purified by immobilized metal ion affinity chromatography (IMAC), essentially as described in U.S. Pat. No. 4,569,794.

Briefly, the IMAC column is prepared as follows. A metal-free chelating resin (e.g. Sepharose 6B IDA, Pharmacia) is washed in distilled water to remove preservative substances and infused with a suitable metal ion [e.g. Ni(II), Co(II), or Cu(II)] by adding a 50 mM metal chloride or metal sulfate aqueous solution until about 75% of the interstitial spaces of the resin are saturated with colored metal ion. The column is then ready to receive a crude cellular extract containing the recombinant protein product.

After removing unbound proteins and other materials by washing the column with any suitable buffer, pH 7.5, the bound protein is eluted in any suitable buffer at pH 4.3, or preferably with an imidizole-containing buffer at pH 7.5.

### EXAMPLE 4

### Detecting Ligands that Bind Dhh-H Using a Chaperonin Protein Assay

The wells of an ELISA plate are coated with chaperonin by incubation for several hours with a 4 ug/ml solution of the protein in Tris-buffered Saline (TBS: 10 mM Tris-HCl, pH7.5, 0.2M NaCl). The plates are then washed 3 times with TBS containing 0.1% Tween-20 (TBST). Then, a mixture of Dhh-H protein (sufficient amount to saturate about 50% of the binding sites on chaperonin) and test compound (10⁻⁹ to 10⁻⁵ M) in about 50 μl volume is added to each well of the plate for an incubation of about 60 minutes. Aliquots of the well solutions are then transferred to the wells of fresh plates and incubated for 60 minutes at room temperature, followed by 3 washes with TBST. Next, about 50 μl of an antibody specific for Dhh-H (SEQ ID NO:2) plus 5% nonfat dry milk are added to each well for a 30 minute incubation at room temperature. After washing, about 50 μl of goat anti-rabbit IgG alkaline phosphatase conjugate at an appropriate dilution in TBST plus 5% nonfat dry milk are added to each will and incubated 30 minutes at room temperature. The plates are washed again with TBST and 0.1 ml of 1 mg/ml p-nitrophenylphosphate in 0.1% diethanolamine is added. Color development (proportional to bound alkaline phosphatase antibody conjugate) is monitored with an ELISA plate reader. When test ligand binding has occurred, ELISA analysis reveals Dhh-H in solution at higher concentrations than in the absence of test ligand.

### EXAMPLE 5

### Production of an Antibody to Dhh-H Protein

Substantially pure Dhh-H protein (e.g. SEQ ID NO:2) or fragment thereof is isolated from transfected or transformed cells using any of the well known methods in the art, or by a method specifically disclosed herein. Concentration of protein in a final preparation is adjusted, for example, by filtration through an Amicon filter device such that the level is about 1 to 5 ug/ml. Monoclonal or polyclonal antibody can be prepared as follows.

Monoclonal antibody can be prepared from murine hybridomas according to the method of Kohler and Milstein (Nature, 256, 495, 1975), or a modified method thereof. Briefly, a mouse is repetitively inoculated with a few micrograms of the protein or fragment thereof, or fusion peptide thereof, over a period of a few weeks. The mouse is then sacrificed and the antibody producing cells of the spleen isolated. The spleen cells are fused by means of polyethylene glycol with mouse myeloma cells. Fused cells that produce antibody are identified by any suitable immunoassay, for example, ELISA, as described in E. Engvall, Meth. Enzymol., 70, 419, 1980.

Polyclonal antiserum can be prepared by well known methods (See e.g. J. Vaitukaitis et.al. Clin. Endocirnol. Metab. 33, 988, 1971) that involve immunizing suitable animals with the proteins, fragments thereof, or fusion proteins thereof, disclosed herein. Small doses (e.g. nanogram amounts) of antigen administered at multiple intradermal sites appears to be the most reliable method.

### EXAMPLE 6

### A Functional Assay for Dhh-H Activity

An assay based on expression of Dhh-H nucleic acids in oocytes to produce a recombinant Dhh-H protein or peptide is carried out as follows. The general method is described in in U.S. Pat. No. 5,585,087, hereby incorporated by reference.

Xenopus laevis oocytes are surgically isolated and manually defolliculated. Defolliculated oocytes are incubated at 18° C. in 1× Normal Amphibian Medium (NAM: 7.5 mM Tris, pH 7.6; 88 mM NaCl 1 mM KCl; 2.4 mM NaHCO[3;]8.2 mM MgSO[4;]0.33 mM Ca(NO3)2; 0.4 mM CaCl2 gentamycin). Defolliculated oocytes are injected with 50 μl cRNA encoding Dhh-H transcripts (approximately 1 μg/μl) or between 0.5-50 ng of cRNA transcripts encoding the secreted Dhh-h protein. Injected oocytes are incubated for 24 hours at 18°-20° C. in 1× (NAM) containing gentamycin.

Grafting of Tissue Explants onto Oocytes.

Xenopus laevis eggs are fertilized and cultured in vitro. At various stages of development, the vitelline membrane is removed and the animal cap(presumptive ectoderm) is explanted from the embryo. The explant is placed in direct contact with a manually defolliculated Xenopus laevis oocyte that had been injected 24 hours previously with Dhh-H cRNA.

Assessment of the Effect of the Recombinant Proteins.

After the explant-oocyte recombinant is cultured for 2 days at 18° C., the embryonic tissue is removed from the oocyte and reverse transcriptase-polymerase chain reaction (RT-PCR) is used to measure the expression levels of various neural and mesodermal marker genes. Alternatively, the recombinant is fixed and stained for marker gene expression by in situ hybridization or by immunostaining.

The assay can also involve the grafting of embryonic tissue that is competent to form mesoderm, or nervous tissue during normal embryonic development, onto oocytes injected with synthetic RNA. If the RNA injected into oocyte encodes a functional Dhh-H protein or fragment thereof, then the grafted ectoderm will differentiate into mesoderm and/or neurotrophic tissues.

### EXAMPLE 7

### Administration of Dhh-H to Mice Enhances Testicular Growth

Male mice were injected daily by subcutaneous route for a two week period with either saline or one of two doses of Dhh-H (SEQ ID NO:2): 2 mg/kg or 10 mg/kg bodyweight. Representative animals were sacrified at days 3, 7 and 14, their tissues extracted, weighed and subjected to histological analysis. The data are summarized in Table 2.

TABLE 2
In vivo effects of human Dhh-H on mouse tissue growth

| | | | Body weight | Body weight | | | | | | |
|---|---|---|---|---|---|---|---|---|---|---|
| Study | | | Initial | Final | KI | LI | HT | SP | BN | TE |
| Day | Dose | Compound | (g) | (g) | (mg) | (g) | (mg) | (mg) | (mg) | (mg) |
| 3 | 0 | Vehicle | 25.3 | 28.9 | 377.7 | 1.2 | 130.3 | 54.7 | 456.0 | x |
| 3 | 2 | FGF-8hb | 24.9 | 26.3 | 353.7 | 1.1 | 145.3 | 62.0 | 436.0 | x |
| 3 | 10 | FGF-8hb | 25.0 | 27.0 | 362.3 | 1.1 | 134.3 | 53.7 | 452.3 | x |
| 3 | 2 | DHH | 25.4 | 26.7 | 359.7 | 1.1 | 148.7 | 62.7 | 457.0 | x |
| 3 | 10 | DHH | 25.1 | 27.7 | 388.3 | 1.1 | 141.0 | 55.3 | 468.0 | x |
| 7 | 0 | Vehicle | 27.6 | 30.0 | 439.3 | 1.3 | 163.7 | 79.7 | 469.3 | x |
| 7 | 2 | FGF-8hb | 26.5 | 29.3 | 439.0 | 1.3 | 148.0 | 69.3 | 450.3 | x |
| 7 | 10 | FGF-8hb | 26.1 | 29.0 | 420.0 | 1.3 | 149.0 | 82.3 | 459.0 | x |
| 7 | 2 | DHH | 24.9 | 26.3 | 375.7 | 1.1 | 162.3 | 51.3 | 436.7 | x |
| 7 | 10 | DHH | 25.2 | 28.3 | 393.7 | 1.2 | 166.3 | 71.0 | 426.3 | x |
| 14 | 0 | Vehicle | 25.5 | 29.5 | 355.0 | 1.0 | 134.0 | 66.7 | 424.7 | 186.0 |
| 14 | 2 | FGF-8hb | 26.1 | 30.9 | 385.0 | 1.2 | 150.7 | 62.3 | 473.3 | 196.3 |
| 14 | 10 | FGF-8hb | 25.0 | 30.4 | 379.3 | 1.1 | 140.7 | 72.3 | 431.0 | 180.3 |
| 14 | 2 | DHH | 26.5 | 31.1 | 409.7 | 1.3 | 137.7 | 75.0 | 475.3 | 216.3 |
| 14 | 10 | DHH | 25.2 | 32.0 | 414.3 | 1.2 | 151.0 | 70.0 | 449.7 | 224.0 |

KI: Kidney; LI: Liver; HT: Heart; SP: Spleen; TE: Testis; BN

We claim:

1. An isolated nucleic acid that hybridizes to SEQ ID NO:3, SEQ ID NO:4, or SEQ ID NO:5 under high stringency conditions.

2. A vector comprising an isolated nucleic acid of claim 1.

3. A host cell containing a vector of claim 2.

4. A pharmaceutical formulation comprising as an active ingredient Dhh-H protein, or fragment thereof associated with one or more pharmaceutically acceptable carriers, excipients, or diluents thereof.

5. A functional analog of Dhh-H that is about at least 80% identical with SEQ ID NO:2 or functional fragment thereof, providing that said analog is not SEQ ID NO:2.

6. A functional analog of Dhh-H that is about at least 90% identical with SEQ ID NO:2 or functional fragment thereof, providing that said analog is not SEQ ID NO:2.

7. A functional analog of Dhh-H that is about at least 95% identical with SEQ ID NO:2 or functional fragment thereof, providing that said analog is not SEQ ID NO:2.

8. A functional fragment of Dhh-H.

9. A method for treating male infertility in humans comprising the administration of a therapeutically effective amount of Dhh-H or functional fragment thereof.

10. A method for treating spinal growth injury in humans comprising the administration of a therapeutically effective amount of Dhh-H or functional fragment thereof.

11. A method for promoting spinal cord regeneration in humans comprising the administration of a therapeutically effective amount of Dhh-H or functional fragment thereof.

12. A method for inhibiting tumor growth in humans comprising the administration of a therapeutically effective amount of Dhh-H or functional fragment thereof.


SEQUENCE LISTING (5 sequences)

SEQ ID NO: 1 - DNA, 1820 bp, Homo sapiens
  CDS (180)..(1367)
  Xaa at position 87 is Phe.
cgctcccggggcacgtgggcagagctagcagcagccaggtgcacggagccacaagagctctagggcactctgggggcagctgtgcctgctgccctctctggtgcctgtggagatgcctaactgacaggagagttagtgaggacaagaacgctcccttttggccgaggtccgctgtatccatggctctcctgaccaatctactgcccctgtgctgcttggcacttctggcgctgccagcccagagctgcgggccgggccgggggccggttggccggcgccgctatgcgcgcaagcagctcgtgccgctactctacaagcaatttgtgcccggcgtgccagagcggaccctgggcgccagtgggccagcggaggggagggtggcaaggggctccgagcgcttccgggacctcgtgcccaactacaaccccgacatcatcttyaaggatgaggagaacagtggagccgaccgcctgatgaccgagcgttgtaaggagcgggtgaacgctttggccattgccgtgatgaacatgtggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctgataactcactggcggtccgggcgggcggctgctttccgggaaatgcaactgtgcgcctgtggagcggcgagcggaaagggctgcgggaactgcaccgcggagactgggttttggcggccgatgcgtcaggccgggtggtgcccacgccggtgctgctcttcctggaccgggacttgcagcgccgggcttcatttgtggctgtggagaccgagtggcctccacgcaaactgttgctcacgccctggcacctggtgtttgccgctcgagggccggcgcccgcgccaggcgactttgcaccggtgttcgcgcgccggctacgcgctggggactcggtgctggcgcccggcggggatgcgcttcggccagcgcgcgtggcccgtgtggcgcgggaggaagccgtgggcgtgttcgcgccgctcaccgcgcacgggacgctgctggtgaacgatgtcctggcctcttgctacgcggttctggagagtcaccagtgggcgcaccgcgcttttgcccccttgagactgctgcacgcgctaggggcgctgctccccggcggggccgtccagccgactggcatgcattggtactctcggctcctctaccgcttagcggaggagctactgggctgagcgtcccaggcatagaaacctcgaagcgcccgaggaaacgagggcctgctggctgagatatggggctgtgggcagcagacgatgccgactggaagggagggagagggagggggagggagaaaatggggctatgcctggcttaggggcaacctcgtactgagaggaggtgataccgggtcctaggtaggtagggctgatggtgcgcactccttaaagaggactatttagcctctcccccagtgcttcagccccacccgataattttattttatttcttatttataaattgtaatataatgattcccttgcccagcttgccaaggtgaggggctggggcatggcattaacactgtctgacacagccagccagtctgacatctgacgttttcctgcaaggtgggctaataaagggaaggctttcaggaaaaaaaaaaaaaaaaaaaaaaaaaa

SEQ ID NO: 2 - protein, 396 aa, Homo sapiens
  Xaa at position 87 is Phe.
MALLTNLLPLCCLALLALPAQSCGPGRGPVGRRRYARKQLVPLLYKQFVPGVPERTLGASGPAEGRVARGSERFRDLVPNYNPDIIXKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPLRLLHALGALLPGGAVQPTGMHWYSRLLYRLAEELLG

SEQ ID NO: 3 - DNA, 1325 bp, Homo sapiens
ggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctgataactcactggcggtccgggcgggcggctgctttccgggaaatgcaactgtgcgcctgtggagcggcgagcggaaagggctgcgggaactgcaccgcggagactgggttttggcggccgatgcgtcaggccgggtggtgcccacgccggtgctgctcttcctggaccgggacttgcagcgccgggcttcatttgtggctgtggagaccgagtggcctccacgcaaactgttgctcacgccctggcacctggtgtttgccgctcgagggccggcgcccgcgccaggcgactttgcaccggtgttcgcgcgccggctacgcgctggggactcggtgctggcgcccggcggggatgcgcttcggccagcgcgcgtggcccgtgtggcgcgggaggaagccgtgggcgtgttcgcgccgctcaccgcgcacgggacgctgctggtgaacgatgtcctggcctcttgctacgcggttctggagagtcaccagtgggcgcaccgcgcttttgcccccttgagactgctgcacgcgctaggggcgctgctccccggcggggccgtccagccgactggcatgcattggtactctcggctcctctaccgcttagcggaggagctactgggctgagcgtcccaggcatagaaacctcgaagcgcccgaggaaacgagggcctgctggctgagatatggggctgtgggcagcagacgatgccgactggaagggagggagagggagggggagggagaaaatggggctatgcctggcttaggggcaacctcgtactgagaggaggtgataccgggtcctaggtaggtagggctgatggtgcgcactccttaaagaggactatttagcctctcccccagtgcttcagccccacccgataattttattttatttcttatttataaattgtaatataatgattcccttgcccagcttgccaaggtgaggggctggggcatggcattaacactgtctgacacagccagccagtctgacatctgacgttttcctgcaaggtgggctaataaagggaaggctttcaggaaaaaaaaaaaaaaaaaaaaaaaaaaagggcggccgccgactagtgagctcgtcgacccggg

SEQ ID NO: 4 - DNA, 397 bp, Homo sapiens
  n at position 24 a pyrimidine;
ctcgtgcccaactacaaccccganatnatnttnaaggatgangagaacagtggagccgaccgcctgatgaccgagcgttgtaaggagcgggtgaacgctttggccattgccgtgatgaacatgtggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctgataactcactggcggtccgggcgggcggctgctttccgggaaatgcaactgtgcgcctgt

SEQ ID NO: 5 - DNA, 306 bp, Homo sapiens
  n at position 282 a purine or pyrimidine
gctggtgaacgatgtcctggcctcttgctacgcggttctggagagtcaccagtgggcgcaccgcgcttttgcccccttgagactgctgcacgcgctaggggcgctgctccccggcggggccgtccagccgactggcatgcattggtactctcggctcctctaccgcttagcgaggagctactgggctgagcgtcccaggcatagaaacctcgaagcgcccgaggaaacgagggcctgctggctgagatatggggctgtgggcagcagacgatgccgactggnaggaggaganggaggggaggagaa